(19) United States
(12) Patent Application Publication
(10) Pub. No.: US 2002/0161336 A1
(43) Pub. Date: Oct. 31, 2002

(54) Needle shield assembly

(75) Inventors: Jamieson William MaClean Crawford, New York, NY (US); Kirk D. Swenson, North Caldwell, NJ (US); C. Mark Newby, Tuxedo, NY (US); Charles G. Hwang, Ridgewood, NJ (US)

Correspondence Address: CASELLA & HESPOS LLP, 274 MADISON AVENUE, SUITE 1703, NEW YORK, NY, 10016-0701, US

(73) Assignee: Becton, Dickinson and Company, Franklin Lakes, NJ (US)

(21) Appl. No.: 10/173,230

(22) Filed: Jun. 14, 2002

Related U.S. Application Data
(63) Continuation-in-part of application No. 09/378,976, filed on Aug. 23, 1999, now Pat. No. 6,440,104.

(51) Int. Cl. A61M 5/32

(52) U.S. Cl. 604/192

(57) ABSTRACT

A medical implement is provided with a hub and a piercing element that projects distally from the hub. The protective cap is removably engaged with the proximal end of the hub. An IV shield is threadedly engaged with the distal end of the hub and protectively covers the piercing element. A hinged shield is hingedly mounted to the hub and can be rotated from a first position substantially adjacent the IV shield to a second position where the hinged shield is spaced rotationally from the IV shield and finally to a third position where the hinged shield encloses the piercing element. The hinged shield must be rotated from the first position to the second position to threadedly disengage the IV shield and to expose the piercing element for use. After use, the hinged shield is rotated from the second position into the third position for protectively enclosing the piercing element.

### RELATED APPLICATION

This application is a continuation-in-part application of U.S. patent application Ser. No. 09/378,976 which was filed Aug. 23, 1999.

### FIELD OF THE INVENTION

The present invention relates to a shield for a needle and more particularly to a safety shield assembly that may be used in conjunction with a syringe assembly, a hypodermic needle, a needle assembly, a needle assembly with a needle holder, a blood collection needle, a blood collection set, an intravenous infusion set or other fluid handing devices or assemblies that contain piercing elements.

### BACKGROUND OF THE INVENTION

Disposable medical devices having piercing elements for administering a medication or withdrawing a fluid, such as hypodermic needles, blood collecting needles, fluid handling needles and assemblies thereof, require safe and convenient handling. The piercing elements include, for example, pointed needle cannula or blunt ended cannula. The piercing element typically is mounted to a hub and extends distally from the hub. A second piercing element may extend proximally from the hub, and may include a non-patient needle that can be placed in communication with a container, such as an evacuated fluid collection container. In other instances, the proximal end of the hub is configured for mating with a medical implement, such as a syringe or a needle holder.

Safe and convenient handling of disposable medical devices is recognized by those in the medical arts so as to minimize exposure to blood borne pathogens. Safe and convenient handling of disposable medical devices results in the disposal of the medical devices intact.

As a result of this recognition, numerous devices have been developed for shielding needles after use. Many of these devices are somewhat complex and costly. In addition, many of these devices are cumbersome to use in performing procedures. Furthermore, some of the devices are so specific that they preclude use of the device in certain procedures or with certain devices and/or assemblies. For example, some devices employ very short thin needle cannulas. A shield designed to lock near the distal end of one needle cannula might not engage a much shorter needle cannula. Additionally, a shield designed to lock with a wider gauge needle cannula might be more likely to generate a spray upon engaging a much narrower needle cannula. Furthermore, it may be desirable to reduce the force required to effect shielding without reducing the audible and tactile indications of complete shielding.

Some medical devices employ a plurality of shields, sleeves and/or caps to achieve sterility and to prevent accidental needle sticks prior to use and to further prevent accidental needle sticks after use. For example, some medical devices employ a rigid generally tubular cap or sleeve telescoped over the piercing element that projects from the distal end of the hub. A second cap or sleeve is telescoped into or over the proximal end of the hub to provide sterility and to prevent accidental sticks with any piercing element that projects from the proximal end of the hub. A hinged shield may be provided on the medical device to prevent accidental sticks with at least the distally directed piercing element after use of the medical device.

Manufacturers of medical devices have preferred methods of use to optimize safe handling, to ensure maximum cleanliness and to avoid accidental sticks. In particular, the user is instructed to remove the proximal cap or sleeve from the hub before removing the sleeve over the piercing element at the distal end of the hub and before manipulating any hinged shield that may be mounted to the hub. The proximal end of the hub then is mounted to the medical device with the distal sleeve in place over the piercing element that projects distally from the hub. Any hinged shield that may be provided on the device then is rotated into a position away from the piercing element, while still keeping the distal sleeve telescoped over the piercing element. The distal sleeve is removed immediately prior to use and is discarded. The medical device then is employed in a specified safe manner. After use, the hinged shield or other such post-use shielding element is moved into a position surrounding the piercing element, and at least portions of the medical device are disposed of in a safe manner.

A need exists for a safety shield assembly: (i) that is manufactured easily; (ii) that is applicable to many devices; (iii) that is simple to use with one hand; (iv) that can be disposed of safely; (v) that does not interfere with normal practices of needle use; (vi) that has tactile features whereby the user may be deterred from contacting the needle, the user may easily orient the needle with the patient and easily actuate and engage the shield assembly; (vii) that has visual features whereby the user may be deterred from contacting the needle, the user may easily orient the needle with the patient and easily actuate and engage the shield assembly; (viii) that is not bulky; (ix) that includes means for minimizing exposure to the user of residual fluid leaking from the needle; and (x) provides minimal exposure to the user because the needle shield is immediately initiated by the user after the needle is withdrawn from the patient's vein. It also would be desirable to provide a safety shield assembly with greater assurance that the preferred unshielding and shielding steps are carried out in a specified safe sequence.

### SUMMARY OF THE INVENTION

The invention relates to a fluid handling device with opposite proximal and distal ends. A piercing element projects at the distal end of the fluid handling device. The piercing element at the distal end of the fluid handling device may comprise a metallic needle cannula, a plastic cannula, a blunt cannula or other piercing element for delivering a fluid to a patient or for obtaining a specimen of fluid. The proximal end of the fluid handling device is configured for communication with another medical implement. For example, the proximal end of the fluid handling device may be configured for mating to a syringe, a fitting on a fluid transfer line, a holder for receiving an evacuated tube or other known medical implement for delivering a fluid to a patient or for obtaining a sample of fluid. In particular, the proximal end of the fluid handling device may define a female Luer fitting. Alternatively, the proximal end of the fluid handling device may comprise a proximal piercing element, such as a non-patient needle intended for communication with an evacuated tube or other medical device.

The fluid handling device further comprises a cap or proximal sleeve mounted over at least portions of the proximal end of the fluid handling device. The cap or proximal sleeve mounted to the proximal end of the fluid handling device has a configuration dependent on the specific structure provided at the proximal end of the fluid handling device. For example, a tubular proximal sleeve may be mounted to the proximal end of the fluid handling device for those instances where the fluid handling device includes a proximal piercing element. Alternatively, an end cap may be mounted to the proximal end of the fluid handling device for those instances where the fluid handling device is configured for mating with a syringe, a fitting or other medical implement. The proximal sleeve or end cap may be frictionally or threadedly mounted to proximal portions of the fluid handling device.

A distal sleeve is telescoped removably to distal portions of the fluid handling device and is configured for protectively covering the piercing element. Proximal portions of the distal sleeve preferably are connected threadedly to the fluid handling device. For example, proximal portions of the distal sleeve may be formed with an array of external threads that can threadedly engage an array of internal threads at or near proximal portions of the distal piercing element. Alternatively, proximal portions of the distal sleeve may include an array of internal threads that engage external threads on the fluid handling device.

The fluid handling device further comprises a hinged safety shield that is intended for shielding the distal piercing element after use. The hinged shield is preliminarily mounted in a position partly surrounding the distal sleeve. However, the dimensions of the distal sleeve prevent the hinged shield from rotating completely over the distal sleeve. The hinged shield is intended to be rotated away from the distal sleeve and away from the piercing element covered by the distal sleeve prior to use of a fluid handling device. After use, the hinged shield is rotated toward the distal piercing element and locks into engagement around the distal piercing element, as explained further herein.

The initial position of the hinged shield in partly surrounding relationship to the distal sleeve prevents or complicates any attempt to threadedly disengage the distal sleeve from the fluid handling device. Hence, the hinged shield must be rotated into the ready-to-use position prior to removal of the distal sleeve. Thus, the fluid handling device inherently ensures that the user of the fluid handling device will follow the preferred safe sequence of first rotating the hinged shield into the ready-to-use position and then removing the distal sleeve. The user is substantially prevented from following the less safe sequence of first removing the distal sleeve and then rotating the hinged shield while the distal piercing element is exposed.

The hinged shield may take many forms. Preferably, the hinged shield comprises a rearward end, a forward end, a slot or longitudinal opening for housing the used needle in the forward end, means for securing the needle in the slot, means for guiding the needle into the slot, means for connecting the hinged shield and the fluid handling device, means for guiding the user's fingers to move the hinged shield into various positions, and means for retaining the hinged shield securely over the used needle.

Desirably, the means for connecting the hinged shield to the fluid handling device is a collar. Preferably, the hinged shield is connected movably to a collar which is connected to a fluid handling device.

Preferably, the hinged shield is connected to the collar by a hanger bar that engages with a hook arm on the collar so that the hinged shield may be pivoted with respect to the collar into several positions. It is within the purview of the present invention to include any structure for connecting the hinged shield to the collar so that the shield may be pivoted with respect to the collar. These structures include known mechanical hinges and various linkages, living hinges, or combinations of hinges and linkages.

Most preferably, the hinged shield is connected to the collar by an interference fit between the hanger bar and the hook bar. Therefore, the shield always is oriented in a stable position and will not move forward or backwards unless movement of the hinged shield relative to the hanger bar and the hook bar is initiated by the user.

Alternatively, the hinged shield and collar may be a unitary one-piece structure. The one-piece structure may be obtained by many methods, including molding the shield and the collar as a one-piece unit, thereby eliminating the separate shield and collar during the manufacturing assembly process.

The assembly of the present invention may further comprise tactile and visual means for deterring the user from contacting the needle, providing easy orientation of the needle with the patient and providing the user with a guide for actuation and engagement with the hinged shield.

The assembly of the present invention may further comprise means for minimizing exposure by the user to residual fluid leaking from a used needle. For example, a polymer material, such as a gel, may be located in the hinged shield.

Most desirably, the assembly of the present invention is such that the cooperating parts of the assembly provide the means for the hinged shield to move into a forward position over the needle. Thus, by simple movement of the hinged shield into a forward position over the used needle, the assembly is ready for subsequent disposal. Therefore, the assembly of the present invention provides minimal exposure of the user to a needle because the shielding is initiated by the user immediately after the needle is withdrawn from the patient's vein.

Desirably, the assembly of the present invention may be used with a syringe assembly, a hypodermic needle, a needle assembly, a needle assembly with a needle holder, a blood collection set, an intravenous infusion set or other fluid handling devices. Preferably, the assembly of the present invention is used with a needle assembly comprising a needle and a hub. Preferably the needle is a conventional double ended needle.

Most preferably, the present invention is used with a needle assembly comprising a hub and a needle connected to the hub whereby the needle comprises a non-patient end and an intravenous end. The collar of the present invention may comprise a hook arm and the hinged shield may be connected movably to the hook arm. Thus the hinged shield may be positioned with respect to the collar and moved easily into several positions.

Preferably, the collar is fitted non-rotatably with the hub of the needle assembly. Additionally, the collar includes cooperating means that mate with reciprocal means on the shield to provide a clear audible and tactile indication of shielding. The cooperating means on the collar may include generally chevron-shaped projection formed on a side of the collar substantially diametrically opposite the hook arm or other such structure that provides the hinge connection to the shield. The chevron-shaped structure includes a forward or distal point. Slanting surfaces diverge and extend proximally from the distal point. The slanting surfaces cooperate with the reciprocal means on the shield to generate a deflection of the sidewalls of the shield away from one another. The chevron-shaped structure further includes proximal ends that are convexly arcuate. The convexly arcuate ends of the chevron-shaped structure on the collar cooperate with the reciprocal means on the shield and with the resiliently deflectable sidewalls of the shield to generate the tactile and audible indication of shielding.

The hinged shield preferably includes at least one cannula finger lock for locked engagement with the cannula when the hinged shield is in the second position around the needle cannula. The cannula finger lock preferably projects obliquely from one sidewall of the hinged shield angularly toward the opposed sidewall and the top wall of the shield. The cannula finger lock is dimensioned, disposed and aligned to contact the needle cannula when the hinged shield approaches the second position. Contact between the cannula and the cannula finger lock will cause the cannula finger lock to resiliently deflect toward the sidewall from which the cannula finger lock extends. Sufficient rotation of the hinged shield will cause the needle cannula to pass the cannula finger lock. As a result, the cannula finger lock will resiliently return to or toward its undeflected condition for securely trapping the needle cannula in the hinged shield.

Preferably, the collar is fitted with the hub of the needle assembly so that the collar cannot rotate around the hub.

Alternatively, the collar and hub may be a unitary one-piece structure. The one piece structure may be accomplished by many methods including molding the collar and the hub as a one-piece unit thereby eliminating the need to separately assemble the collar to the hub during the manufacturing process.

Most preferably, the collar is fitted with the hub of the needle assembly so that the bevel surface or bevel up surface of the intravenous or distal end of the needle faces the same side of the collar when the hinged shield is in the open position. Alignment of the collar, hub, hinged shield and needle with the bevel surface up makes it easier to insert the needle into the patient without manipulating the assembly. The orientation of the intravenous end of the needle with the bevel up assures the user that the needle is properly oriented for use and does not require any manipulation before use. Most notably, the orientation of the hinged shield provides a visual indication to the user of the orientation of the bevel surface of the needle.

Preferably, the hinged shield is capable of pivoting from a first position, where the intravenous end of the needle is exposed and bevel up, to an intermediate position where the needle is partially covered, to a second position where the needle is contained by the shield.

Alternatively, it is within the purview of the present invention that the hinged shield, collar and hub is a unitary one-piece structure. The one-piece structure may be accomplished by many methods including molding the hinged shield, collar and hub as a one-piece unit thereby eliminating the need to separately assemble the hinged shield, collar and hub during the manufacturing process.

It is an advantage of the present invention that the hinged shield covering the used intravenous end of the needle provides easy containment of the used needle. A further advantage of the hinged shield is that it will only move upon initiation by the user.

The assembly of the present invention when used with a fluid handling device is also easily disposable when removed from a conventional needle holder, or other such device.

A notable attribute of the present invention is that it is easily adaptable with many devices. For example, the invention is usable with syringe assemblies, hypodermic needles, needle holders, blood collection needles, blood collection sets, intravenous infusion sets such as catheters or other fluid handling devices or assemblies that contain piercing elements.

Another notable attribute of the present invention is that the tactile and visual features deter the user from touching the needle, allow the user to easily orient the needle with the patient and guide the user to actuate and engage the shield of the assembly.

### BRIEF DESCRIPTION OF THE DRAWINGS

FIG. 1 is a perspective view of the safety shield assembly of the present invention as connected to a needle assembly and related packaging features.

FIG. 2 is a perspective view of the unassembled pieces of FIG. 1.

FIGS. 3A and 3B are bottom views of the shield as shown in FIG. 2.

FIG. 4 is a cross sectional view of the collar as shown in of FIG. 2 taken along lines 4-4 thereof.

FIG. 5 is a cross sectional view of the needle hub as shown in FIG. 2 taken along lines 5-5 thereof.

FIG. 6 is a cross sectional view of the shield of FIG. 2 taken along lines 6-6 thereof.

FIGS. 7-12 illustrate the use of the safety shield assembly with the needle assembly of FIG. 1 with a conventional needle holder.

FIG. 13 is a cross sectional view of the assemblies in use with a conventional needle holder as shown in FIG. 12 taken along lines 13-13 thereof.

FIG. 14 is a cross-sectional view of the assemblies of FIG. 13 taken along lines 14-14 thereof.

FIG. 15 is a bottom view of the assemblies as shown in FIG. 11.

FIG. 16 illustrates an additional embodiment of the present invention, whereby a gel material is located in the shield as shown in a bottom view of the assemblies of FIG. 11.

FIG. 17 is a perspective view of an additional embodiment of the present invention in use with a blood collection set.

FIG. 18A is an exploded perspective view of an additional embodiment of the present invention intended for use with a syringe.

FIG. 18B is a perspective view of the collar of the embodiment of FIG. 18A.

FIG. 18C is a side elevational view of the embodiment of FIG. 18A mounted to a syringe.

FIG. 19 is a perspective view of an additional embodiment of the present invention in use with a catheter.

### DETAILED DESCRIPTION OF THE INVENTION

While this invention is satisfied by embodiments in many different forms, there is shown in the drawings and will herein be described in detail, the preferred embodiments of the invention, with the understanding that the present disclosure is to be considered as exemplary of the principles of the invention and is not intended to limit the invention to the embodiments illustrated. Various other modifications will be apparent to and readily made by those skilled in the art without departing from the scope and spirit of the invention. The scope of the invention will be measured by the appended claims and their equivalents.

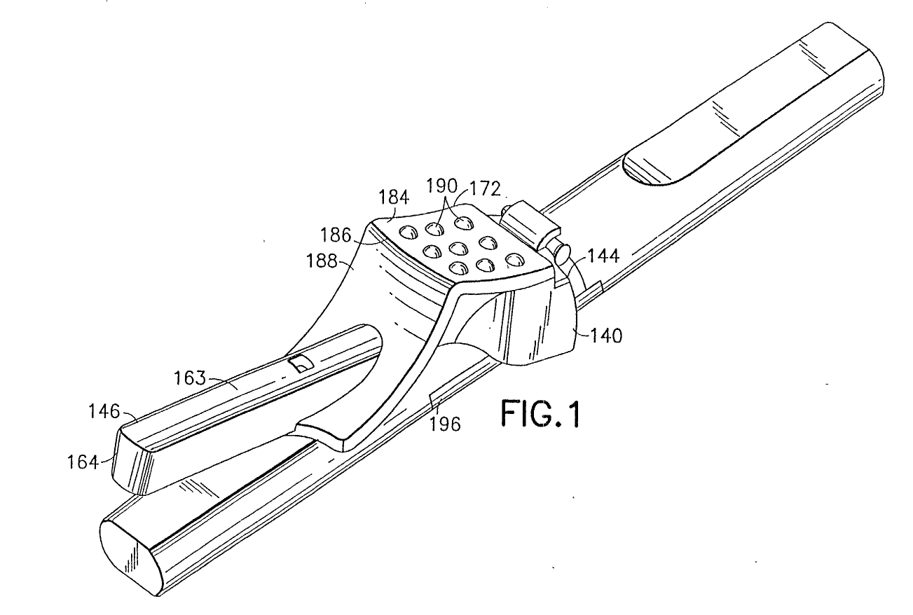

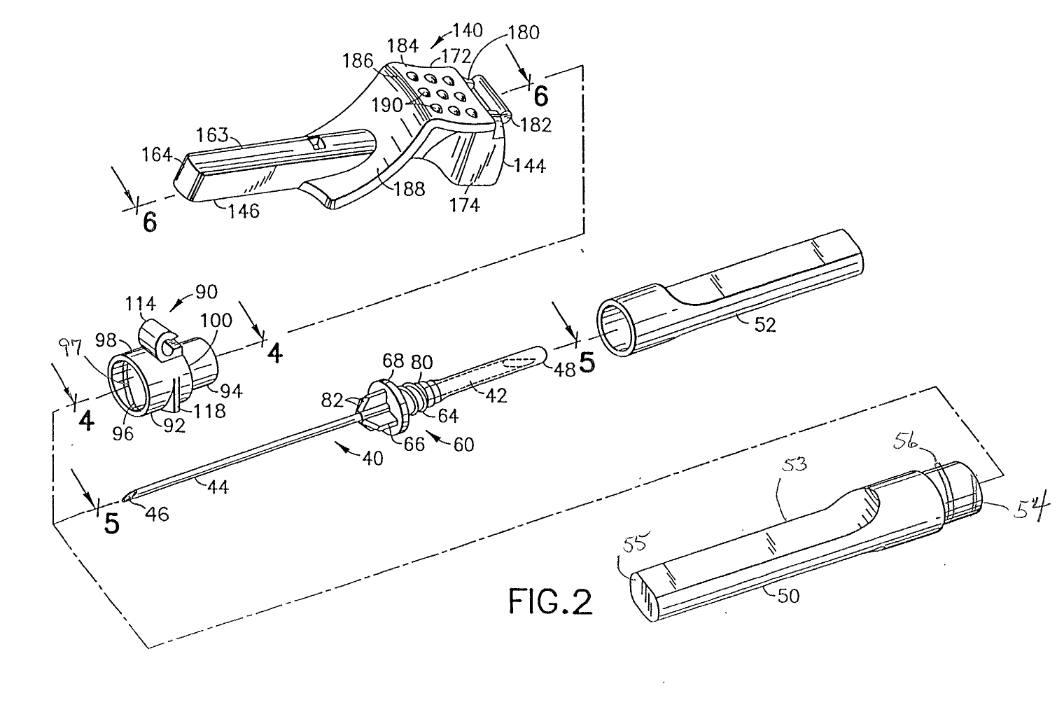

Referring to the drawings in which like reference characters refer to like parts throughout the several views thereof, FIGS. 1 and 2 illustrate a needle assembly with the safety shield assembly of the present invention and the related packaging features. The needle assembly includes a needle 40, a hub 60, packaging features to cover the needle and a label. The safety shield assembly includes a collar 90 and a hinged shield 140.

As shown in FIGS. 2 and 5, needle 40 includes a non-patient end 42, an intravenous end 44 and a passageway 46 extending between the non-patient end and the intravenous end. An elastomeric sleeve 48 covers the non-patient end. A first rigid sleeve 50 covers the intravenous end and a second rigid sleeve 52 covers both the non-patient end and the elastomeric sleeve. As shown in FIG. 1, a label 196 may also be applied to the finally assembled parts. First rigid sleeve 50 includes a rigid tubular sidewall 53 with an open proximal end 54 and a closed distal end 55. An array of external threads 56 extends around first rigid sleeve 50 adjacent proximal end 54.

As shown in FIGS. 2 and 5, hub 60 includes a threaded end 64, a ribbed end 66 and passageway 62 extending between the threaded end and the ribbed end. Threaded end 64 and ribbed end 66 are separated by flange 68. Non-patient end 42 of needle 40 extends from threaded end 64 and intravenous end 44 of needle 40 extends from ribbed end 66. Preferably, threaded end 64 comprises male threads 80 for mounting the hub on a conventional needle holder and ribbed end 66 comprises male ribs 82 for connecting the hub and collar 90.

As shown in FIGS. 2 and 4, collar 90 includes a forward skirt 92 and a rearward skirt 94. Forward skirt 92 is cylindrical and comprises an inner circumferential surface 96 with an array of internal threads 97 and an outer circumferential surface 98. Forward skirt 92 mates with rearward skirt 94 at a shoulder 100. Rearward skirt 94 is cylindrical and comprises an inner circumferential surface 102 and an outer circumferential surface 104 and extends from shoulder 100 opposite of forward skirt 92. The inner diameter of forward skirt 92 is larger than the inner diameter of rearward skirt 94. Alternatively, the inner diameters for collar 90 can be equal. A hook 114 extends from outer circumferential surface 98 of forward skirt 92. Additionally a chevron-shaped protrusion 118 projects outwardly from outer circumferential surface 98 of forward skirt 92 at a side opposite hook 114. The chevron-shape protrusion 118 is substantially symmetrically formed and has a peak 120 pointed toward forward skirt 92 and ramp surfaces 122 that diverge symmetrically from peak 120 toward rearward skirt 94. Ramp surfaces 122 terminate at rounded ends 124 at the outer side and proximal extremes of chevron-shaped protrusion 118. Rounded ends 124 extend continuously into the proximal side of chevron-shaped protrusion 118 facing toward rearward skirt 94.

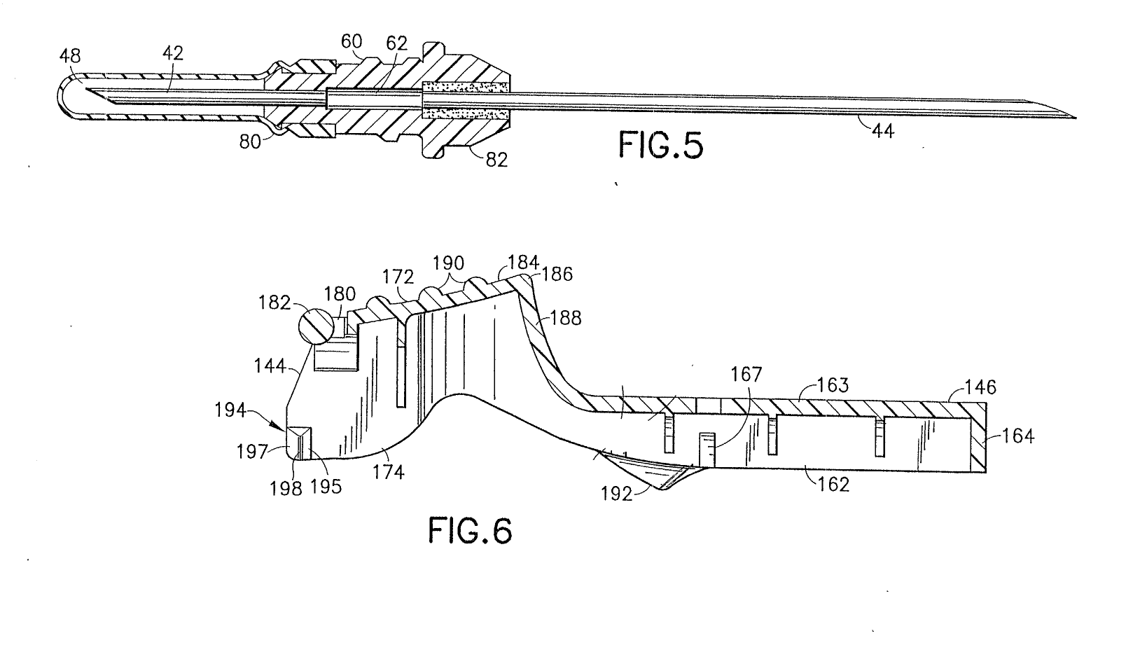

As shown in FIGS. 2 and 6, hinged shield 140 comprises a rearward end 144 and a forward end 146.

Forward end 146 of hinged shield 140 includes a slot or longitudinal opening 160 formed by sidewalls 162 that extend downwardly from top wall 163 and run substantially opposite of one another in parallel along the length of slot 160 towards forward end wall 164. Slot 160 is slightly wider than needle 40. Sidewalls 162 include bottom edges 165 that extend substantially parallel to one another and parallel to top wall 163.

A cannula finger lock 167 is located at one of sidewalls 162 and is configured to secure the used needle. Cannula finger lock 167 extends from a location on a first of the sidewalls 162 adjacent the bottom edge 165 thereof and projects angularly toward the opposed sidewall 162 and toward the top wall 163. The projection of the cannula finger lock 167 from the respective sidewall 162 preferably exceeds half the distance between the respective sidewalls. Cannula finger lock 167 is deflectable by the needle when the needle enters slot 160. Once the needle passes the end of cannula finger lock 167, the cannula finger lock moves back to its original position so that the needle is permanently trapped in slot 160 by cannula finger lock 167.

Rearward end 144 of hinged shield 140 defines a collar engaging area 166 that is a continuation of slot 160. Collar engaging area 166 includes a rearward end 168, a forward end 170, a top finger guide area 172, sidewalls 174 that extend downwardly from top finger guide area 172, an underside area 176 dimensioned for surrounding collar 90, and extending arms 180 to support and hold hanger bar 182. Sidewalls 174 are spaced apart by a major width adjacent rearward end 168. The major width is selected to enable sidewalls 174 to slide across diametrically opposite side surfaces of forward skirt 92 of collar 90. Sidewalls 174 converge, however, toward forward end 170 to define a minor distance therebetween substantially equal to the distance between sidewalls 162 at forward end 146 of hinged shield 140. Sidewalls 174 include bottom edges 177 that face away from top finger guide area 172. As shown most clearly in FIG. 6, bottom edges 177 curve toward top finger guide area 172 at locations between rearward end 168 and forward end 170 of collar engaging area 166.

The extreme rear ends of sidewalls 174 on collar engaging area 166 include rounded ears 194 that project toward one another from opposed inner surfaces 175 of sidewalls 174. Rounded ears 194 are disposed to engage chevron-shaped protrusion 118 on collar 90. More particularly, each rounded ear 194 includes a distal surface 195, a proximal surface 197 and a curved surface 198 extending between distal and proximal surfaces 195 and 197. Distal surface 194 is aligned to sidewall 174 at an angle of approximately 60° and proximal surface 197 is aligned to sidewall 174 at an angle of approximately 45°. Curved surface 198 extends smoothly and convexly between distal and proximal surfaces 195 and 197. Proximal surfaces 197 of rounded ears 194 will engage ramp surfaces 122 of chevron-shaped protrusion 118 to deflect sidewalls 174 slightly away from one another as hinged shield 140 approaches the second position. This deflection of sidewalls 174 will occur substantially simultaneously with the deflection of cannula finger lock 167. The apex of curved surface 198 on each rounded ear 194 passes the respective rounded proximal end surface 124 on chevron-shaped projection 118 on collar 90 slightly before cannula finger lock 167 passes the needle cannula. As a result, sidewalls 174 begin to return resiliently toward an undeflected condition. This resilient return of sidewalls 174 cooperates with raked distal surfaces 195 on rounded ears 194 to cause sidewalls 174 to snap against chevron-shaped projection 118. This snapping action provides a clear audible and tactile indication of complete shielding and occurs substantially when the used needle is trapped by cannula finger lock 167. The angles of distal and proximal surfaces 195 and 197 of rounded ears 194 affects the performance of hinged shield 140. In particular, a smaller acute angle alignment of proximal face 197 reduces the force required to move hinged shield 140 past rounded ears 194. A larger acute angle proximal surface 197 of rounded ears 194 requires a greater force to move hinged shield 140 toward the second position. Similarly, the angle between distal surface 195 and sidewall 174 affects the acceleration characteristics as hinged shield 140 is propelled toward the second position in response to the resilient return of sidewalls 174. This change in acceleration characteristics affects the audible indication of shielding.

Top finger guide area 172 comprises a first ramp 184 that extends slightly on an upwardly slope from the rearward end of the collar engaging area to a shoulder 186. From shoulder 186 extends a second ramp 188 which slopes downwardly towards top section 163. Most preferably, first ramp 184 comprises touch bumps 190. The touch bumps provide a tactile and visual guide to alert the user that the user's finger has contacted the shield and that the shield is in a defined or controlled position. The touch bumps may be any configuration so long as they extend and are distinct from the top finger guide area. The touch bumps may also be of a distinguishing color as compared to the top finger guide area or the shield.

Second ramp 188 has interior surface 192 for urging the needle toward the center of slot 160 as the shield is being rotated into the closed position. The exterior surfaces are slightly inclined and extending radially from the second ramp. The interior surfaces are especially helpful if the longitudinal axis of the needle is misaligned with respect to the longitudinal axis of the hub.

Extending arms 180 are located at rearward end 168 and at the beginning of top finger area 172 and hold hanger bar 182.

The safety shield assembly and the needle assembly are assembled together whereby needle 40 is connected to hub 60 and sealed with adhesive at the ends of the hub. Hub 60 then is joined with collar 90 by ultra-sonic welding techniques or any other bonding techniques, or mechanical fit, whereby rearward annular skirt 94 of collar 90 mates with ribbed end 66 of the hub. Male ribs 82 of the hub are contained or forced fitted within inner sidewall 102 of rearward annular skirt 94 of collar 90. Collar 90 is aligned with the intravenous end of needle 40 whereby the hook 114 is aligned with the bevel up of needle 40. External threads 96 adjacent proximal end 54 of first rigid sleeve 50 then are threaded into engagement with internal threads 97 formed on inner circumferential surface 96 of forward skirt 92 of collar 90 to cover needle 40. Thereafter, hinged shield 140 is connected to collar 90 whereby hanger bar 182 is force fitted into hook 114 whereby slot 160 faces first rigid sleeve 50. Most preferably, hinged shield 140 is connected to the collar by a force fit or interference fit between hanger bar 182 and hook 114. Therefore, hinged shield 140 is always oriented in a stable position and will not move unless movement of the shield is positively initiated by the user. To assemble the last piece, shield 140 is moved towards rigid sleeve 50 and second rigid sleeve 52 is force fitted onto outer sidewall 104 of rearward skirt 94 of collar 90.

In addition, a label 196 may be applied to the finally assembled parts. The label may be used to provide tamper resistance of the parts, so that they are not reused.

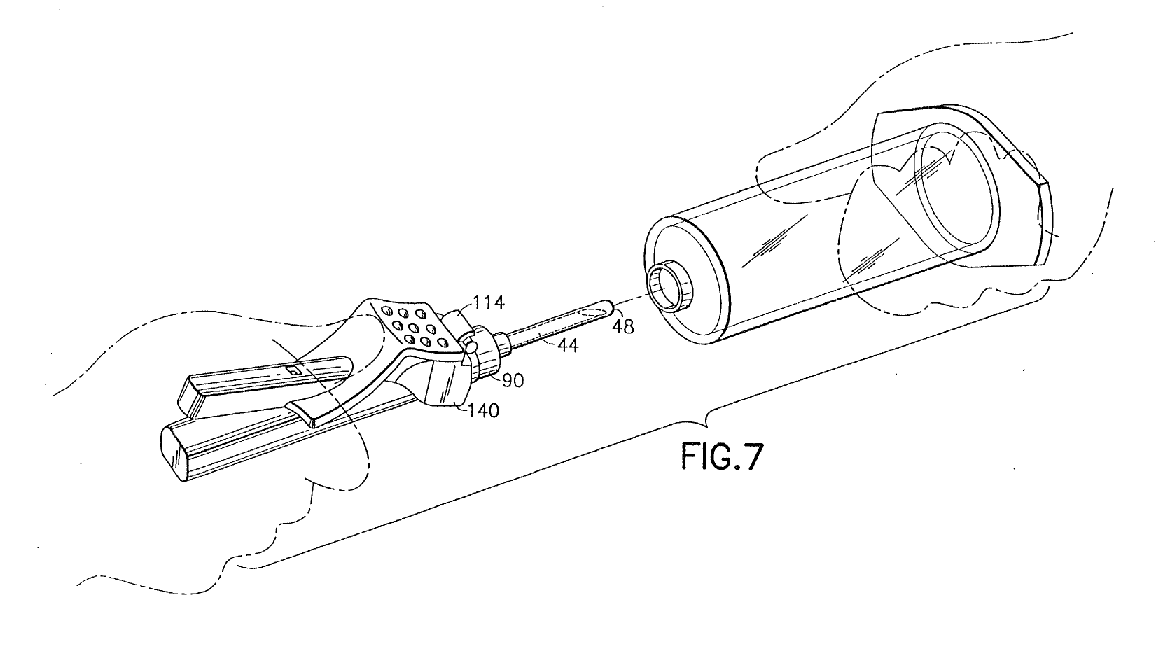

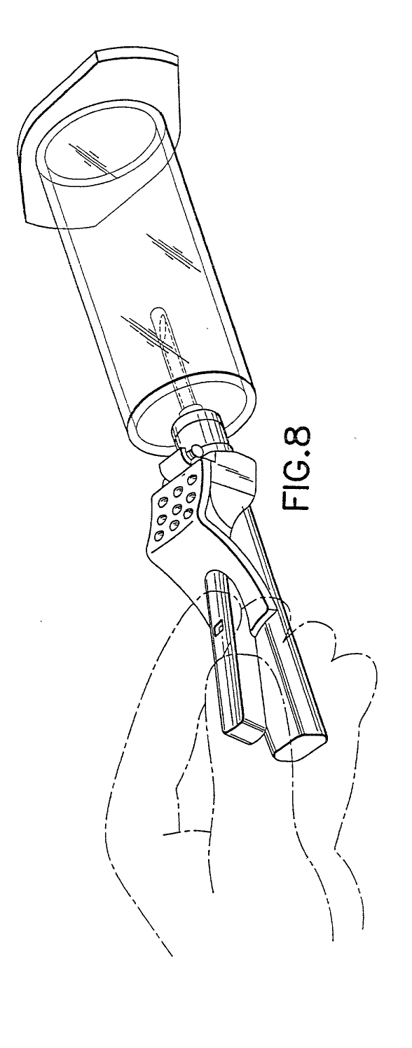

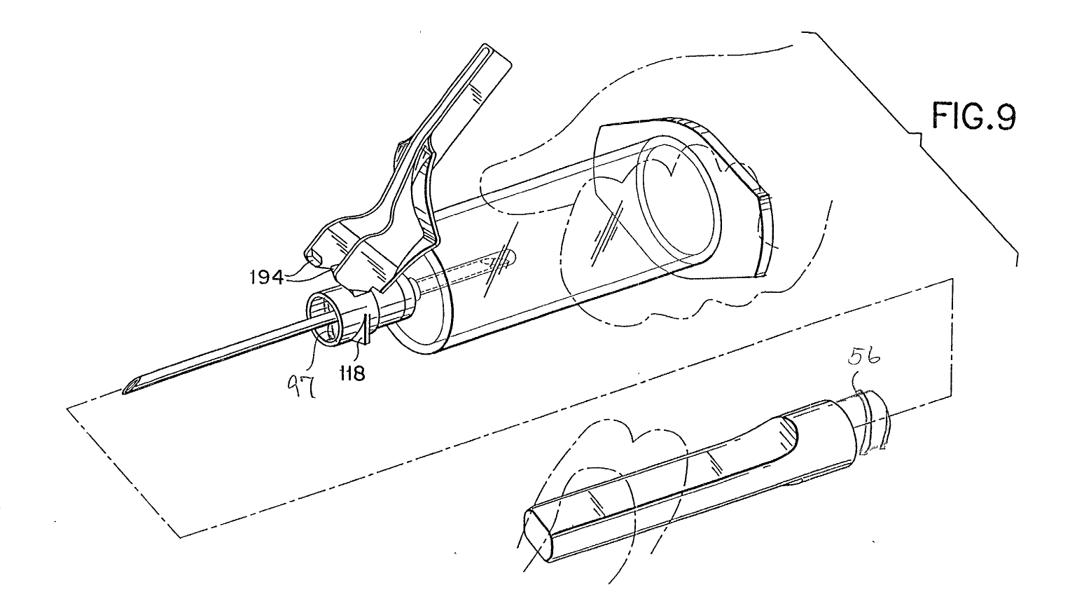

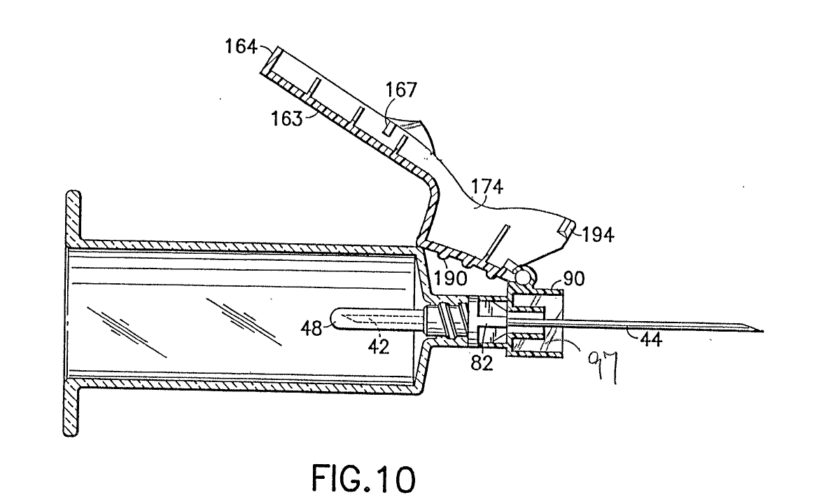

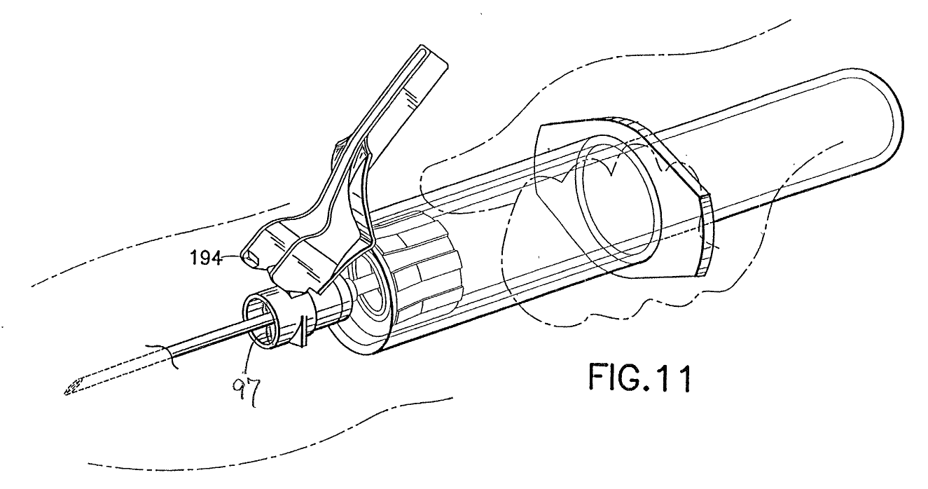

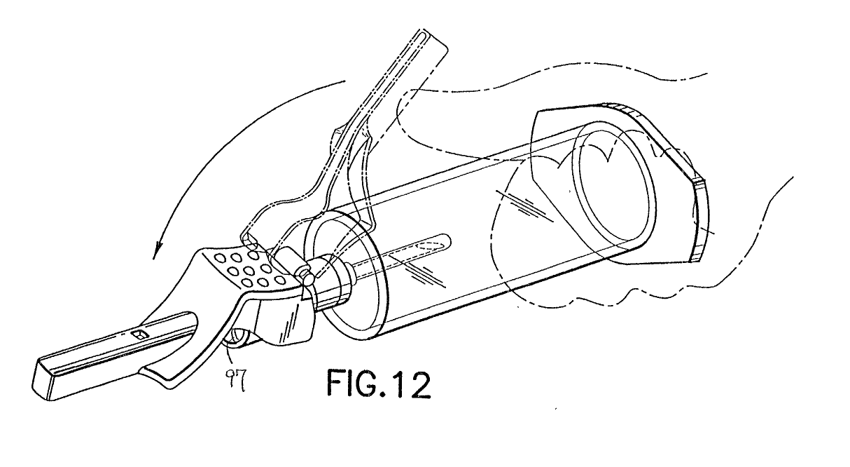

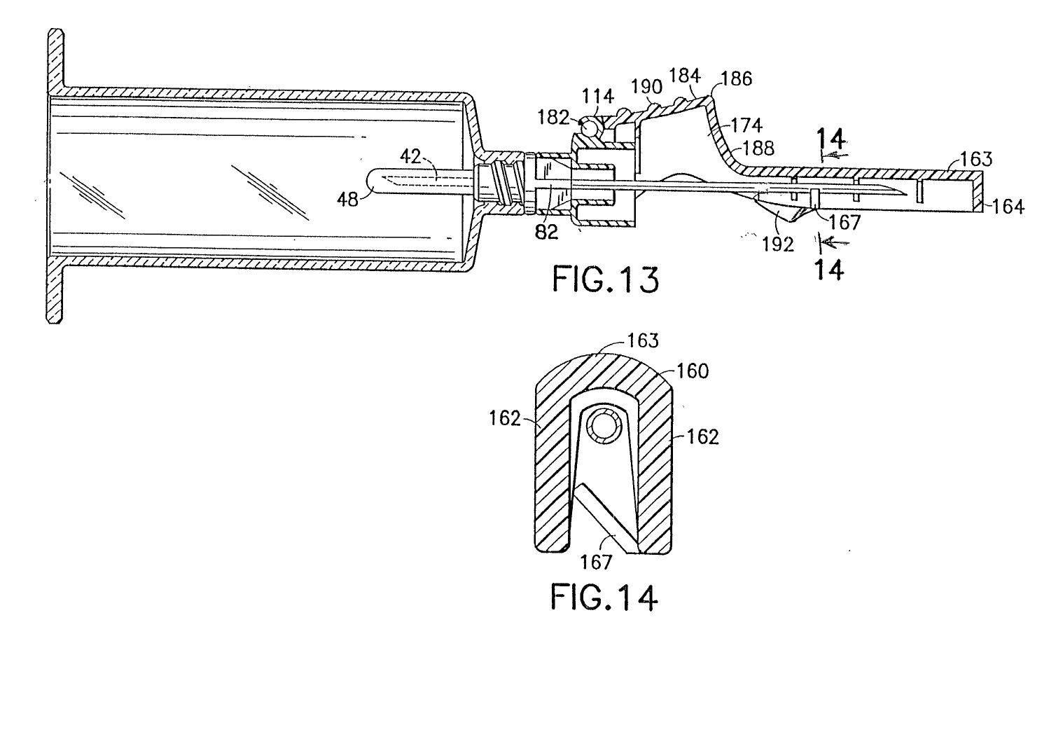

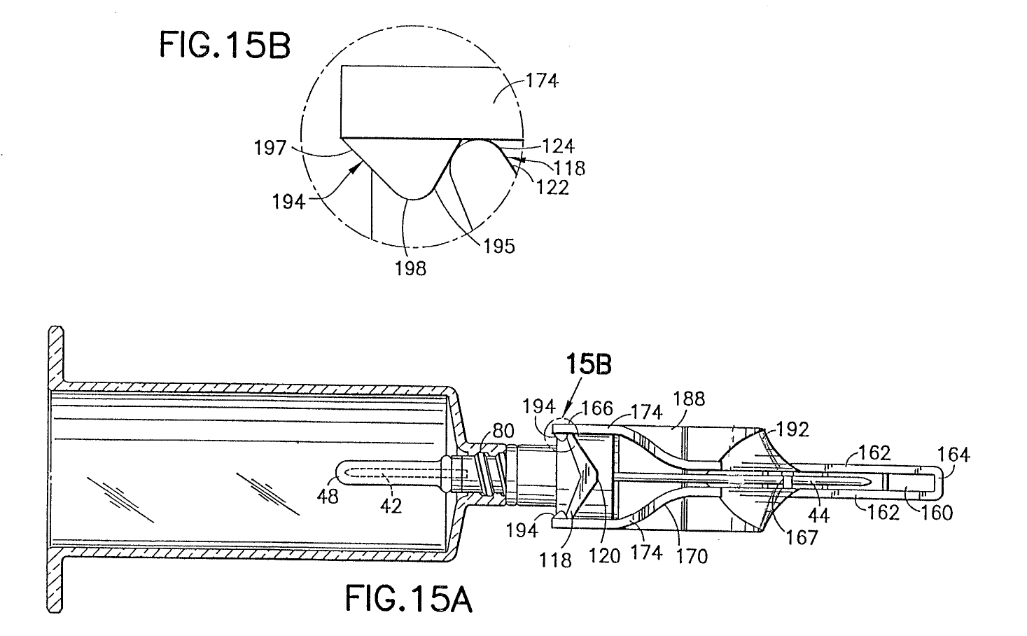

In use, as shown in FIGS. 7-15, second rigid sleeve 52 is removed from the non-patient needle by pulling proximally on second rigid sleeve 52. A slight twisting force may be required to tear label 196. A needle holder then is screwed onto threads 64 of hub 60. As specifically shown in FIGS. 9 and 10, hinged shield 140 then is rotated back by the user towards the needle holder and first rigid sleeve 50 is threadedly disengaged from forward skirt 92 of collar 90 to remove the covering from the intravenous needle. Then as shown in FIG. 11, a venipuncture is conducted whereby the intravenous end of the needle is inserted into a vein of a patient and an evacuated tube having a closure is inserted into the needle holder. Then as shown in FIGS. 12-15, when the venipuncture is complete the user easily rotates hinged shield 140 from the open position towards the intravenous needle to an intermediate position and then the user pushes on the shield at the top finger guide area to move the shield into a second position whereby the needle is trapped in the longitudinal opening. More particularly, needle 40 contacts cannula finger lock 167. The engagement of needle 40 with cannula finger lock 167 causes cannula finger lock 167 to deflect toward top wall and toward the sidewall 162 from which cannula finger lock 167 projects. Sufficient rotation of hinged shield 140 will cause needle 40 to pass cannula finger lock 167. As a result, cannula finger lock 167 will return resiliently to an undeflected condition. Thus, needle 40 will be trapped above cannula finger lock 167.

Needle 44 is contained within hinged shield 140 as the shield is pivoted into the second position. More particularly, proximal surfaces 197 of rounded ears 194 move over detents 118 and cause sidewalls 174 to deflect away from one another. The angularly aligned proximal faces 197 of rounded ears 194 ensure easy movement of shield 140. Additionally, the resiliency of sidewalls 174 and the angular alignment of distal surface 195 of ears 194 causes hinged shield 140 to be accelerated into the second position. This accelerated movement of shield 140 helps to generate a clear audible and tactile indication of shielding.

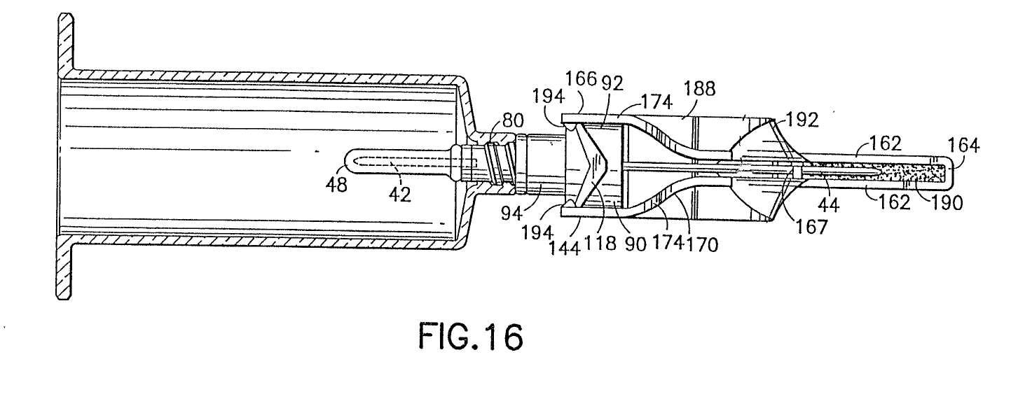

Alternatively as shown in FIG. 16, a gel material 190 is located in hinged shield 140 so that when the needle snaps past cannula finger lock 167 it will come to rest in gel material 190. The gel material will contain any residual fluid that may be on the needle. Simultaneously, rounded ears or projections 198 move over detents 118. This causes sidewalls 174 to deflect away from one another and then to snap back into engagement with collar 90 to provide a clear audible and tactile indication of complete shielding.

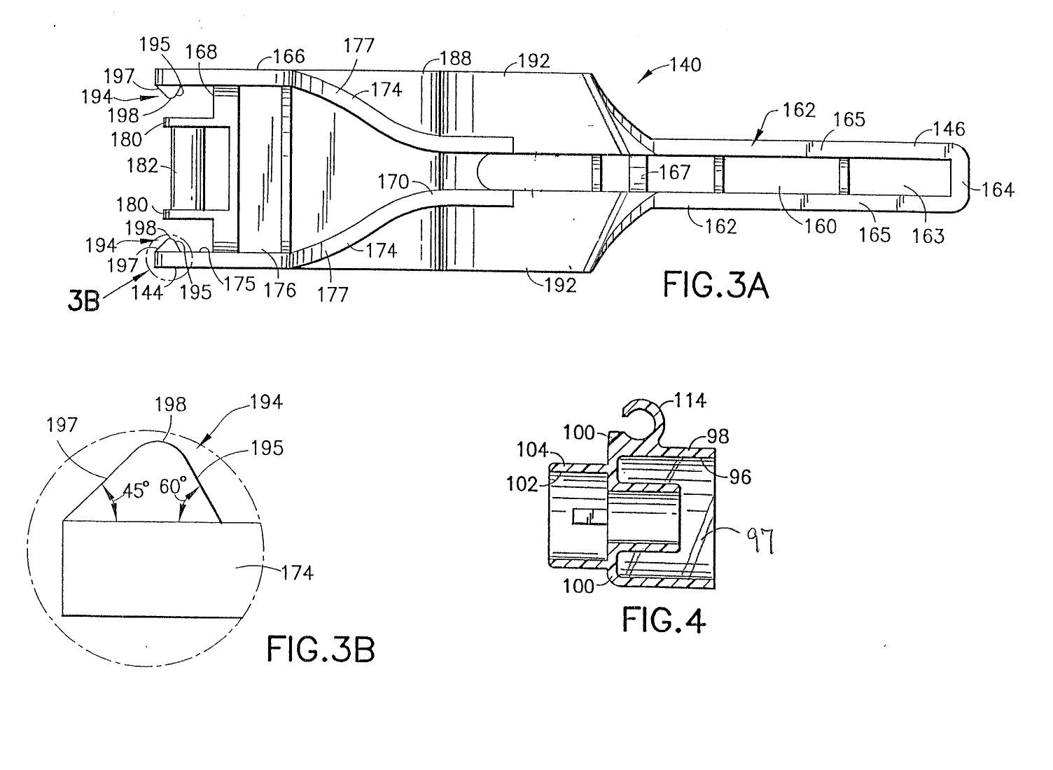

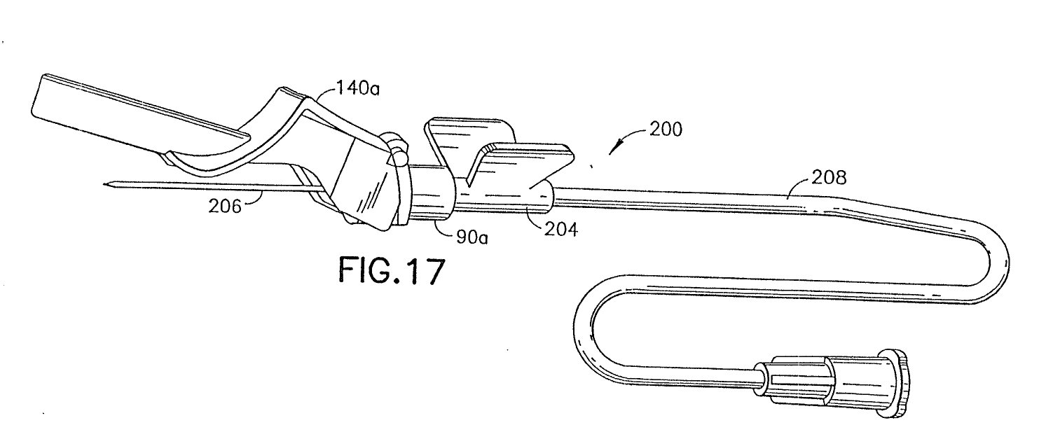

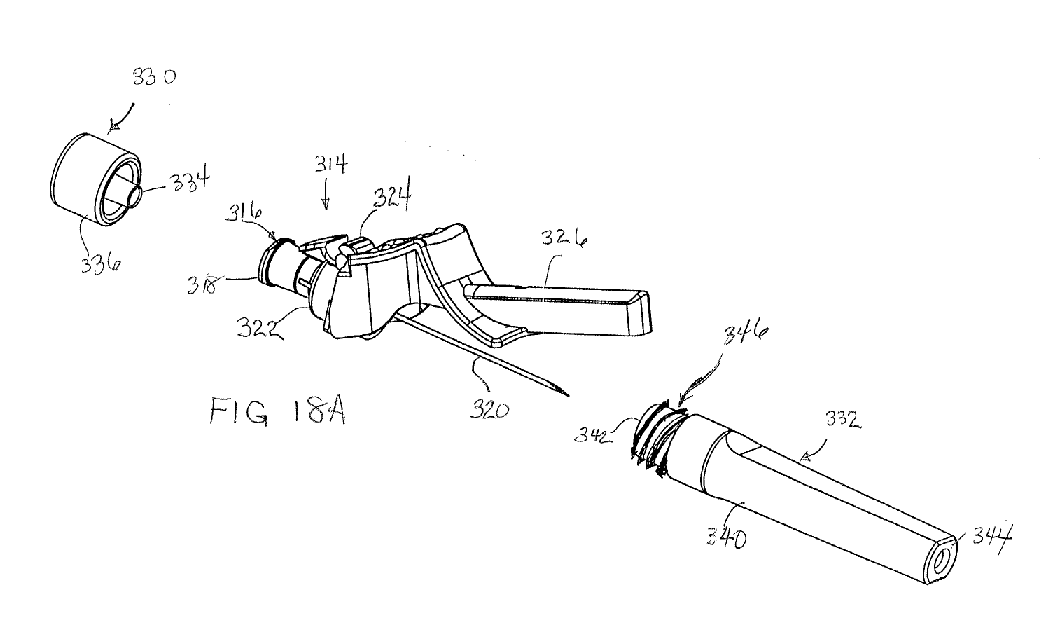

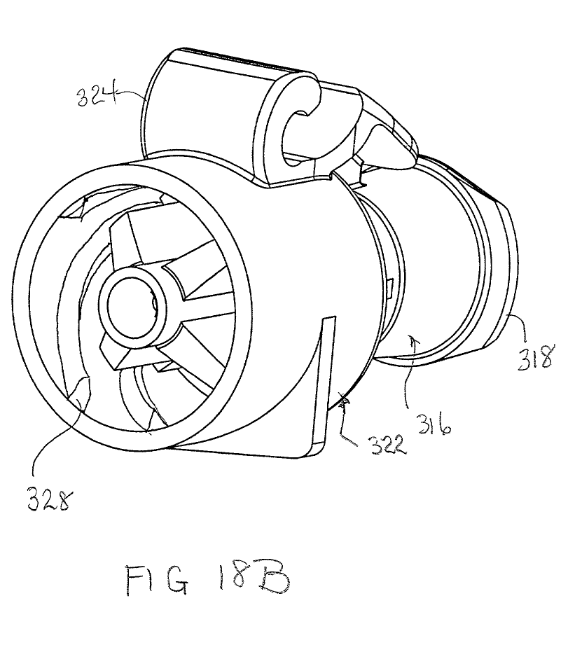

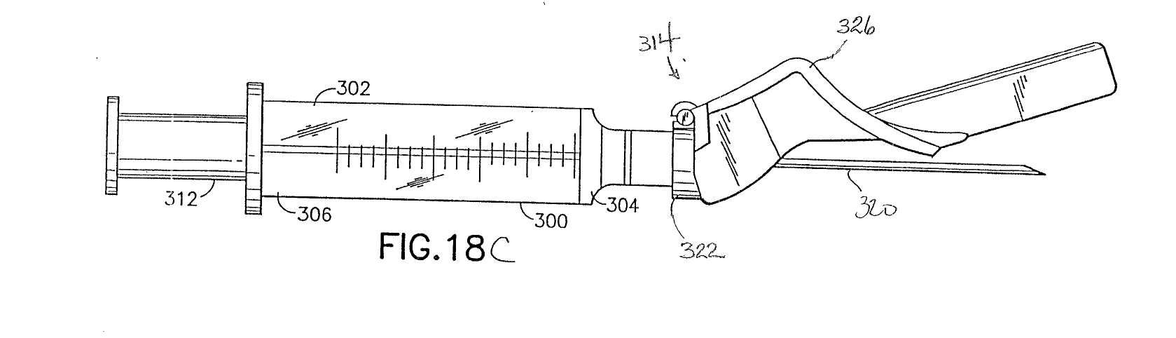

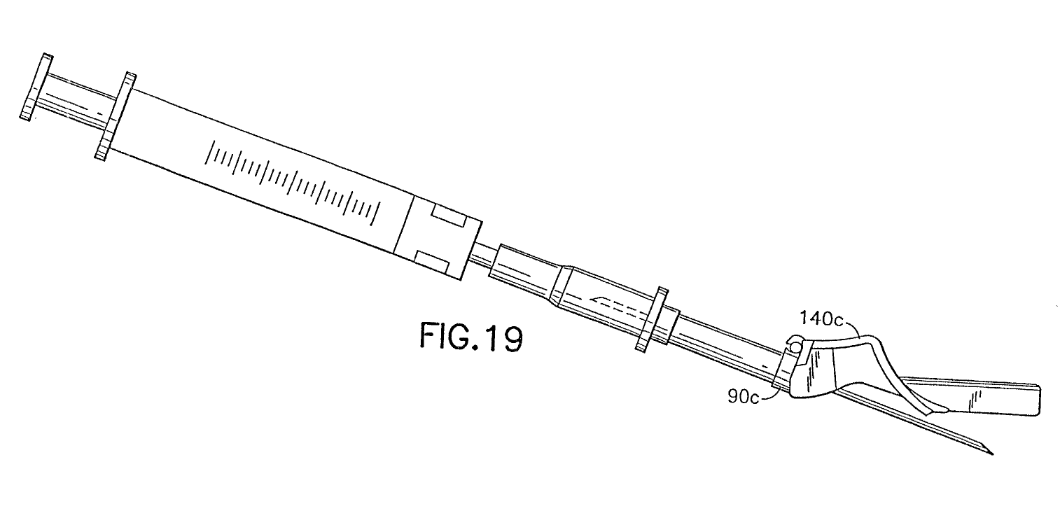

FIGS. 17, 18A-C, and 19 are further embodiments of the invention that may include components which are substantially identical to the components of FIGS. 1-3. Accordingly, similar components performing similar functions will be numbered identically to those components of FIGS. 1-3, except that a suffix “a” will be used to identify those similar components in FIG. 17, a suffix “b” will be used to identify those similar components in FIGS. 18A-C and a suffix “c” will be used to identify those similar components in FIG. 19.

For purposes of illustration, hinged shield 140a and collar 90a are connected to a conventional IV infusion set, 200, or butterfly structure comprising a needle body with a needle hub 204 extending from the forward end of the needle body and a needle 206 embedded in hub 204. Extending from the rearward end of the needle body is flexible tubing 208 which is conventional and utilized to allow the user to manipulate the structure and to connect it subsequently to supplies of infusion liquids or for the return of collected blood if the arrangement is being used to collect blood.

Infusion set 200 further comprises flexible wings 210 attached to and projecting outwardly from needle hub 204.

Alternatively, the safety shield assembly of the present invention may be used in conjunction with a syringe, as illustrated in FIGS. 18A-C.

For purposes of illustration a conventional hypodermic syringe 300 comprises a syringe barrel 302 having a distal end 304, a proximal end 306 and a plunger 312. In this embodiment, a needle assembly 314 includes a hub 316 with a proximal end 318 that defines a female Luer fitting that can be mate with distal end 304 of syringe barrel 302. An intravenous needle 320 projects distally from hub 316. A collar 322 is mounted rigidly to hub 316 and includes a hook 324 at a location aligned substantially with the bevel up side of intravenous needle 320. Alternatively, collar 322 and hub 316 may be a single component. A shield 326 is hingedly mounted to hook 324. Collar 322 and hinged shield 326 are substantially identical to embodiments described and illustrated in greater detail above. In particular, collar 322 is provided with an array of internal threads 328, as shown in FIG. 18B.

Pre-use sterility and safety are maintained by an end cap 330 and an IV shield 332. End cap 330 includes a male Luer projection 334 and an outer collar 336. Male Luer projection 334 is dimensioned to be frictionally retained within female Luer fitting at proximal end 318 of hub 316. Outer collar 336 is dimensioned to be frictionally retained around hub 316. End cap 330 can be removed from hub 316 with an exertion of proximally directed axially forces that may be combined with a slight rotational twisting force relative to hub 316. End cap 330 prevents contamination of interior portions of hub 316, and hence also prevents contamination of the lumen through intravenous needle 320. IV shield 332 comprises a rigid generally tubular sidewall 340 with a proximal end 342 and a closed distal end 344. Outer surface regions of IV shield 332 adjacent proximal end 342 define an array of external threads 346 that are dimensioned for threaded engagement with internal threads 328 on collar 322. Thus, the IV shield can be threadedly mounted to collar 322 for protectively covering IV needle 320 and further contributing to sterility of IV needle 320.

Prior to use, end cap 330 is mounted frictionally over proximal portions of needle hub 316 and IV shield 332 is mounted threadedly to internal threads 328 of collar 322 and over intravenous needle 320. Hinged shield 326 then is rotated into a partly closed condition where proximal portions of hinged shield 322 partly surround and frictionally engage portions of IV shield 322 distally of and adjacent to external threads 346.

The needle assembly is used by initially separating end cap 330 from needle hub 316. Threaded engagement of IV shield 332 ensures that IV shield 332 will not inadvertently become separated from collar 322 in response to axial pulling forces exerted on end cap 330. Thus, IV needle 320 remains safely covered and protected. Proximal end 318 of needle hub 316 then is mounted to distal end 304 of syringe 300. IV shield 332 must be removed to access needle 320 and to use syringe 300. The removal of IV shield 332 requires the disengagement of external threads 346 on IV shield 332 from internal threads 328 on collar 322. However, the initial disposition of hinged shield 326 partly surrounding and adjacent IV shield 332 substantially prevents IV shield 332 from being threadedly disengaged from collar 322 without first rotating hinged shield 326 away from IV shield 332 and into the ready-to-use position. Thus, the user must follow the preferred practice of rotating hinged shield 326 away from needle cannula 320 and into the ready-to-use position prior to threadedly disengaging IV shield 332. Accordingly, the needle assembly of FIGS. 18A-C substantially prevents the less safe practice of first removing IV shield 332 to expose needle 320 and then manually moving hinged shield 326 while the intravenous needle 320 is exposed. Furthermore, the needle assembly shown in FIGS. 18A-C ensures that the end cap 330 will be removed before exposing needle 320. Accordingly, hub 316 is likely to be threadedly engaged with syringe 300 before rotating hinged shield 326 into the ready-to-use position and before separating IV shield 332. FIGS. 18A-C show a threaded connection between IV shield 332 and collar 322. However, other attachment mechanisms can be provided between IV shield 332 and collar 322 that would make separation difficult while hinged shield 326 is in partly surrounding disposition to IV shield 332. For example, detents can be provided between IV shield 332 and collar 322 that would make simple pulling of IV shield 332 away from collar 322 difficult. The detent may require some rotational movement of IV shield relative to collar 322 to overcome frictional interference. Other such connections that would require secure gripping of IV shield 332 and/or twisting of IV shield 332 to effect removal may be provided.

Alternatively, the present invention may be used in conjunction with a catheter as illustrated in FIG. 19.

The shield and collar of the safety shield assembly of the present invention are comprised of moldable parts which can be mass produced from a variety of materials including, for example, polyethylene, polyvinyl chloride, polystyrene or polyethylene and the like. Materials will be selected which will provide the proper covering and support for the structure of the invention in its use, but which will provide also a degree of resiliency for the purpose of providing the cooperative movement relative to the shield and the collar of the assembly.

The illustrated embodiments show the first rigid sleeve or IV shield with external threads and the hub with the mating internal threads. However, the relative disposition of the internal and external threads may be reversed.

The illustrated embodiments show a cannula finger lock for engaging the needle. However, other means may be provided for maintaining the hinged shield around the needle, including more than one cannula finger lock or differently configured needle engaging structures.

What is claimed is:

1. A medical implement comprising:
a hub having opposite proximal and distal ends;
a piercing element projecting from said distal end of said hub;
a protective cap removably mounted to said proximal end of said hub; and
an IV shield threadedly engaged with said distal end of said hub and protectively covering said piercing element, and a hinged shield hingedly mounted to said hub and selectively rotatable between a first position where said hinged shield lies substantially adjacent said IV shield, a second position where said hinged shield is rotated away from said IV shield and a third position where said hinged shield lockingly surrounds said piercing element, whereby said hinged shield must be rotated from said first position to said second position for threadedly disengaging said IV shield from said hub for exposing said piercing element.

2. The medical implement of claim 1, wherein said hub includes an array of internal threads, said IV shield comprising a proximal end with an array of external threads threadedly engaged with said internal threads of said hub.

3. The medical implement of claim 2, wherein said IV shield is a substantially tubular structure with a rigid sidewall extending distally beyond said piercing element.

4. The medical implement of claim 1, wherein said piercing element is a metallic needle cannula having a proximal end permanently mounted to said distal end of said hub and a sharply pointed distal end remote from said hub.

5. The medical implement of claim 1, wherein said distal end of said hub includes an array of external threads, and wherein said IV shield includes a proximal end with an array of internal threads threadedly engaged with said external threads of said hub.

6. The medical implement of claim 1, wherein the protective cap is frictionally retained with said proximal end of said hub.

7. The medical implement of claim 6, wherein said proximal end of said hub defines a female Luer fitting, and wherein said cap comprises a male projection frictionally engaged with said female Luer fitting of said hub.

8. The medical implement of claim 1, wherein said protective cap is threadedly engaged with said proximal end of said hub.

9. The medical implement of claim 1, wherein said hinged shield comprises at least one resiliently deflectable lock for permanent locked engagement with said piercing element.

10. The medical implement of claim 1, wherein the hinged shield partly surrounds the IV shield when the hinge shield is in the first position.

11. The medical implement of claim 1, further comprising a holder for receiving an evacuated fluid collection tube, said holder being engageable with said hub.

12. The medical implement of claim 1, further comprising a syringe releasably engagement with said hub.

13. The medical implement of claim 1, further comprising a medical IV infusion set releasably engagement with said hub.

14. A medical implement comprising a hub having opposite proximal and distal ends, a piercing element projecting from said distal end of said hub;
an IV shield threadably engaged with said distal end of said hub and protectively covering said piercing element, and a hinged shield hingedly mounted to said hub and selectively rotatable between a first position where said hinged shield lies substantially adjacent said IV shield, a second position where said hinged shield is rotated away from said IV shield and a third position where said hinged shield lockingly surrounds said piercing element; and
a tamper evidence tab with an adhesive surface, said tab adhered to said IV shield and at least one of said hub and said hinged shield, wherein rotating said IV shield from said hub breaks said tamper evidence tab.

15. A medical implement comprising:
a hub having opposite proximal and distal ends;
a piercing element projecting from said distal end of said hub; and
an IV shield threadedly engaged with said distal end of said hub and protectively covering said piercing element, and a hinged shield hingedly mounted to said hub and selectively rotatable between a first position where said hinged shield lies substantially adjacent said IV shield, a second position where said hinged shield is rotated away from said IV shield and a third position where said hinged shield lockingly surrounds said piercing element, whereby said hinged shield must be rotated from said first position to said second position for threadedly disengaging said IV shield from said hub for exposing said piercing element.
